(19) United States
(12) Patent Application Publication
(10) Pub. No.: US 2013/0140447 A1
(43) Pub. Date: Jun. 6, 2013

(54) Gantry positioning device and imaging device using the same

(71) Applicants: Duck-June Kim, Gyeonggi-do (KR); Sang-Hoon Ji, Seoul (KR); Woong-Hee Shon, Gyeonggi-do (KR)

(72) Inventors: Duck-June Kim, Gyeonggi-do (KR); Sang-Hoon Ji, Seoul (KR); Woong-Hee Shon, Gyeonggi-do (KR)

(73) Assignee: Korea Institute of Industrial Technology, Chungcheonganam-do (KR)

(21) Appl. No.: 13/384,915

(22) PCT Filed: Oct. 12, 2011
(86) PCT No.: PCT/KR2011/007571

§ 371 Date: Dec. 3, 2012

(30) Foreign Application Priority Data
May 25, 2011 (KR) 10-2011-0049398

(51) Int. Cl. A61B 6/00; G01J 1/02; F16M 11/18

(52) U.S. Cl. CPC A61B 6/4447; F16M 11/18; G01J 1/02
USPC 250/234; 248/371

(57) ABSTRACT

A gantry positioning device for connecting a gantry and a supporter together of an imaging system includes: a linear translation positioner for translating the gantry vertically with respect to the supporter; a first rotation positioner for rotating the gantry about a first axis which passes horizontally an iso-center of the gantry with respect to the supporter; and a second rotation positioner for rotating the gantry about a second axis which passes vertically the iso-center of the gantry with respect to the supporter. The first rotation positioner includes: a tilt guide which is formed along an arc having a center on the first axis; and a tilt block which is connected to the tilt guide so as to be rotatable about the first axis.

### CROSS-REFERENCE TO RELATED APPLICATION

This application claims priority to and the benefit of Korean Patent Application No. 10-2011-0049398 filed in the Korean Intellectual Property Office on May 25, 2011, the entire contents of which are incorporated herein by reference.

### FIELD OF THE INVENTION

The present invention relates to a gantry positioning device for regulating positions of a gantry for an imaging system and an imaging device using the same.

### BACKGROUND ART

An imaging device which has a gantry positioning device of five degree of freedom which can control position of a gantry through linear translations along three directions and rotations about two axis was introduced(referring to US patent U.S. Pat. No. 7,338,207).

In this conventional imaging device, the gantry positioning device supports the gantry in a cantilever type, so multi-axis motions more than three degree of freedom should be used in order to realize a wag motion which an iso-center is maintained at a specific point.

FIG. 1 is a schematic diagram showing multi-axis motions for realizing wag motion which maintains an iso-center of a gantry in a conventional imaging device.

Referring to (a) of FIG. 1, if a wag motion is realized by rotating the gantry by degree a (using motion of one degree of freedom) using a gantry positioning device (not shown) of cantilever type, iso-centers A and B of the gantry are not fixed but move by dz and dx. Accordingly, by reversely moving the gantry by dx and dz (additionally using motion of two degree of freedom), the iso-centers A and B can be fixed (i.e., A=B) (referring to (b) and (c) of FIG. 1).

As such, it is difficult for the conventional gantry positioning device to maintain the iso-center through simple motions. In addition, the gantry is supported by a cantilever type at a side of a horizontal direction of the gantry (direction of dx in FIG. 1), so high initial cost is needed to obtain a desired stability and careful maintenance is needed, and by the horizontal connection overall space of the imaging device is relatively great.

### Technical Problem

The present invention has been made in an effort to provide an imaging a gantry positioning device and an imaging device in which a tilt motion and a wag motion of a gantry can be easily performed without multi-axis motion.

### Technical Solution

A gantry positioning device for connecting a gantry and a supporter together of an imaging system according to an embodiment of the present invention includes: a linear translation positioner for translating the gantry vertically with respect to the supporter; a first rotation positioner for rotating the gantry about a first axis which passes horizontally an iso-center of the gantry with respect to the supporter; and a second rotation positioner for rotating the gantry about a second axis which passes vertically the iso-center of the gantry with respect to the supporter. The first rotation positioner includes: a tilt guide which is formed along an arc having a center on the first axis; and a tilt block which is connected to the tilt guide so as to be rotatable about the first axis.

The second rotation positioner may connects the gantry and the tilt block such that the gantry is rotatable about the second axis, the horizontal direction may be a horizontal direction with respect to the supporter, and the vertical direction may be a vertical direction with respect to the gantry.

The second rotation positioner may include: a wag guide which is formed to the first rotation positioner for guiding a rotation about the second axis; and a wag block which is connected to the wag guide so as to be rotatable about the second axis and is connected to the gantry.

The linear translation positioner may include the first rotation positioner and the supporter together such that the gantry is movable along the vertical direction.

The linear translation positioner may include: a linear translation guide which is provided to the supporter to guide a movement along the vertical direction; and a linear translation block which is connected to the linear translation guide so as to be movable along the vertical direction and to which the tilt guide is provided.

The linear translation block may include a tilt power transmission unit for transmitting power for rotating the tilt block along the tilt guide to the tilt block, and the tilt block comprises a tilt portion which receives the power.

The tilt power transmission unit may include a rotation gear for transmitting the power, and the tilt portion comprises a tilt gear which is engaged with the rotation gear to receive the power so that the tilt block rotates about the first axis.

An imaging device according to an embodiment of the present invention includes a gantry positioning device according to one of the embodiments of the present invention, a gantry and a supporter.

The gantry may include a gantry operation unit, and the gantry operation unit may include a light source emitting light and a light detector receiving the light, and the light source and the detector are disposed to face each other.

The supporter may be movable.

### Advantageous Effects

According to the present invention, by the first rotation positioner and the second rotation positioner, a tilt motion and a wag motion of a gantry can be realized by one-axis motion using one freedom of degree in a state that an iso-center of a gantry is fixed at one point, and accordingly, emission of light to a user being scanned can be performed in an iso-centric state in which an operation center is maintained at one point while a tilt motion or a wag motion is being performed, so precise and quick scanning from various directions can be stably performed.

Further, a linear translation of a gantry along a vertical direction can be freely made by a linear translation positioner, an user being scanned can be easily set at an operation center.

Further, since a gantry is positioned while being supported from the bottom by a gantry positioning device, stability in an operation can be enhanced, when compared to a conventional type in which the gantry is supported in a cantilevered type, so initial cost for securing the stability or maintenance cost can be reduced, and in terms of space, total space of the device can be substantially reduced compared to the conventional cantilevered type which has a lateral connection so that spatial utilization can be improved.

Further, since a supporter is movable, an user being scanned can be easily set at an operation center by the combination of a vertical linear translation by a linear translation positioner.

### BRIEF DESCRIPTION OF DRAWINGS

FIG. 1 is a schematic diagram showing multi-axis motions for realizing wag motion which maintains an iso-center of a gantry in a conventional imaging device.

FIG. 2 is a perspective view of an imaging device including a gantry positioning device according to an embodiment of the present invention.

FIG. 3a and FIG. 3b are drawings showing an example of a tilt motion for tilting a gantry of a gantry positioning device of FIG. 2.

FIG. 4 is an enlarged view of a portion depicted by a dotted line in FIG. 3b.

FIG. 5a and FIG. 5b are drawings showing an example of a wag motion for waging a gantry of a gantry positioning device of FIG. 2.

FIG. 6 is an enlarged view of a portion depicted by a dotted line in FIG. 5b.

FIG. 7a and FIG. 7b are drawings showing an example of a linear translation for translating vertically a gantry of a gantry positioning device of FIG. 2.

FIG. 8 is an enlarged view of a portion depicted by a dotted line in FIG. 7b.

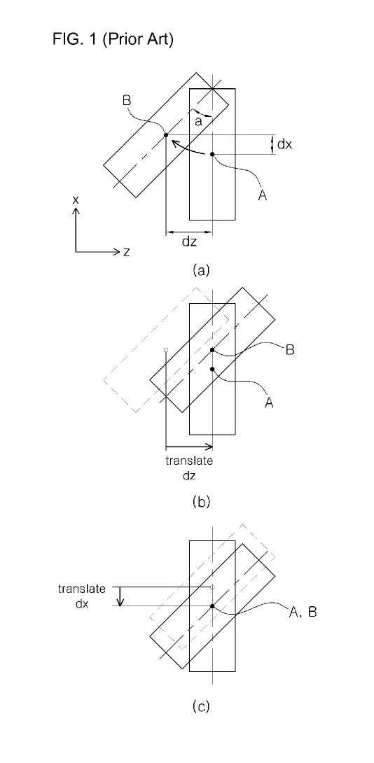

### DETAILED DESCRIPTION OF THE EMBODIMENTS

Embodiments of the present invention will be explained in detail with reference to the attached drawings.

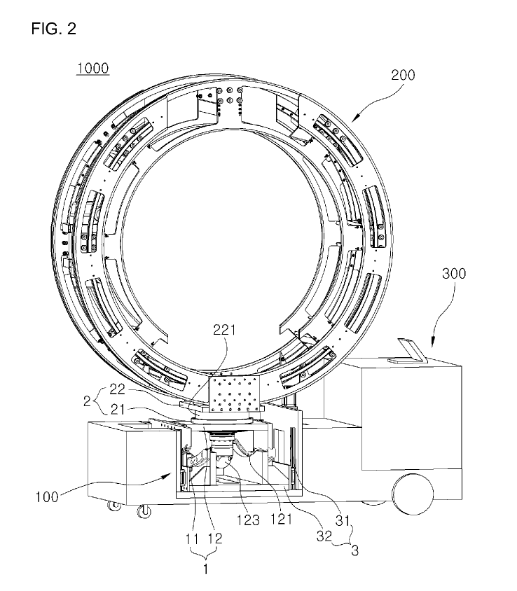

FIG. 2 is a perspective view of an imaging device including a gantry positioning device according to an embodiment of the present invention.

Referring to FIG. 2, a gantry positioning device 100 according to an embodiment of the present invention relates to a gantry positioning which connects a gantry 200 of an imaging system to a supporter 300. In more detail, the gantry positioning device 100 relates to a gantry positioning which connects the gantry 200 of an imaging system to the supporter 300 in such a way that the gantry 200 may undergo various translations and rotations while maintaining an iso-center in an imaging device 1000 taking two-dimensional or three-dimensional images by a rotation of the gantry 200 in a circumferential direction.

The gantry positioning device 100 will be explained hereinafter.

First, a first rotation positioner 1 will be explained.

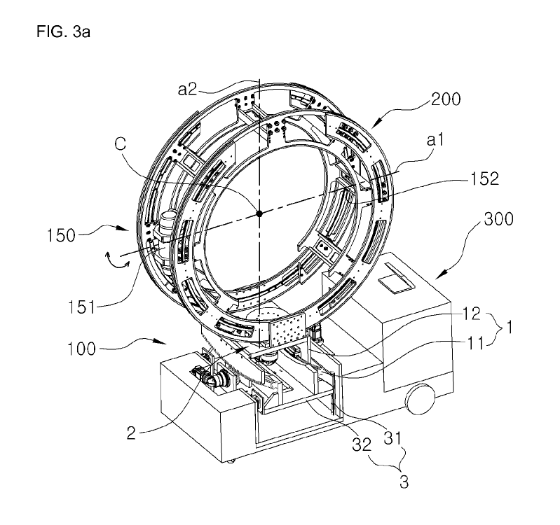

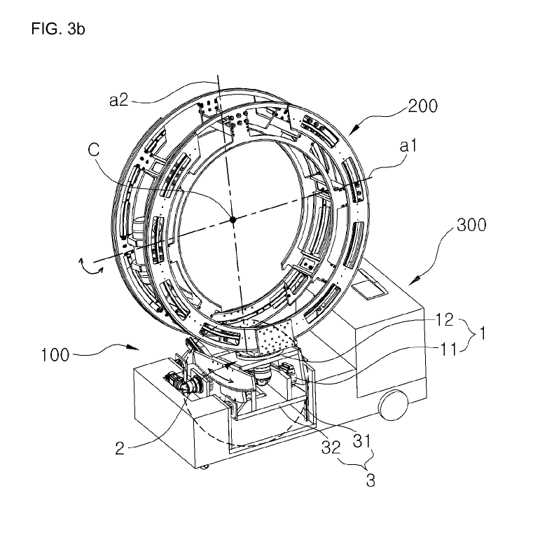

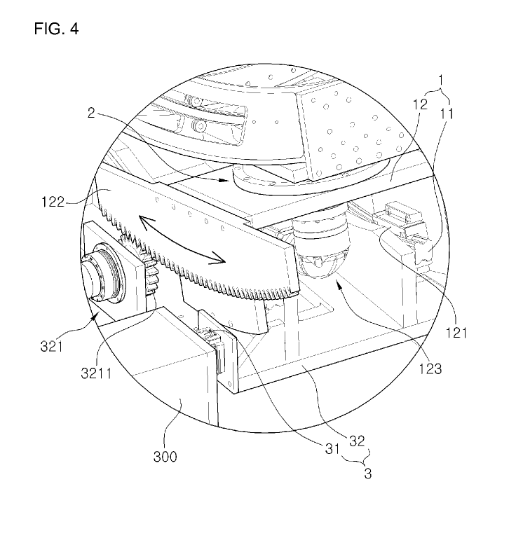

FIG. 3a and FIG. 3b are drawings showing an example of a tilt motion for tilting a gantry of a gantry positioning device of FIG. 2, and FIG. 4 is an enlarged view of a portion depicted by a dotted line in FIG. 3b.

Referring to FIG. 3a and FIG. 3b, the first rotation positioner 1 rotates the gantry 200 about a first axis a1 with respect to the supporter 300. At this time, the first axis a1 may be an axis penetrating an iso-center C of the gantry 200 in a horizontal direction. At this time, a horizontal direction may be a horizontal direction al with respect to the supporter 300. In more detail, the horizontal direction al with respect to the supporter 300 may be a direction parallel with a horizontal radial direction of the ring-shaped gantry 200 in the imaging device 1000 in a state of non-tilted or non-wagged of the gantry 200 as shown in FIG. 2.

That is, as shown in FIG. 3a and FIG. 3b, a tilt motion of the gantry 200 can be realized by the first rotation positioner 1. In addition, the tilt motion is realized by the rotation of the gantry 200 about the first axis a1, and the first axis a1 passes the iso-center C of the gantry 200 as above-described, so the iso-center C of the gantry 200 may be fixed while the tilt motion is made. That is, through the first rotation positioner 1, the gantry 200 may be tilted by one-axis motion using one freedom of degree while the iso-center C does not move but maintains an iso-centric state.

For reference, the iso-center C may be a center of a volume which an imaging process is performed by the gantry 200. For example, the iso-center C may be a cross-sectional point of a rotation axis of the gantry 200 for a rotation in a circumferential direction and a center route of light projection of a light source to a light detector.

Referring to FIG. 3a, FIG. 3b, and FIG. 4, the first rotation positioner 1 may include a tilt guide 11 which is formed along an arc about the first axis a1, and a tilt block 12 which is connected to the tilt guide 11 so as to be rotatable about the first axis a1.

Exemplarily, referring to FIG. 4, the tilt guide 11 may be a curved rail which is formed along an arc of a circle having a center on the first axis a1. In addition, referring to FIG. 4, the tilt block 12 may include a tile coupling portion 121 which is engaged with the tilt guide 11 to be movable along the tilt guide 11.

For example, by the engagement of the tilt block 12 with the tilt guide 11 via the tile coupling portion 121, the tilt block 12 may undergo a tilt motion about the first axis a1 within a predetermined range allowed by a range of the tilt guide 11. The tilt motion by the engagement of the tilt guide 11 and the tilt block 12 may also be realized in the gantry 200 which is connected by a second rotation positioner 2. In addition, since the first axis a1 is an axis passing the iso-center C of the gantry 200, the tilt motion of the gantry 200 can be realized by one-axis motion using one freedom of degree which the iso-center C of the gantry 200 is fixed at one point.

Exemplarily, referring to FIG. 3a, FIG. 3b, and FIG. 4, in configuration of the tilt block 12 and the tilt guide 11, the tilt guide 11 is formed in a linear translation block 32 of a linear positioner 3, so the first rotation positioner 1 is rotatably connected to the linear positioner 3 via connections with the tilt block 12 and the tilt guide 11 so as to have connections for realizing the tilt motion.

However, the first rotation positioner 1 is not limited to these dispositions and connections. That is, to the contrary, the tilt coupling portion 121 of the tilt block 12, instead of the tilt guide 11, is disposed to the linear translation block 32, and the first rotation positioner 1 may be formed such that the tilt guide 11 is connected to the tilt coupling portion 121. In this case, the tilt block 12 may have connections of being engaged with the tilt guide 11 via the tilt coupling portion 121 which is disposed to the linear translation block 32 in a state that the tilt guide 11 is directly mounted thereto. In other words, dispositions of lower configurations such as male-female connections for driving the first rotation positioner 1 may be changed with each other for necessities.

For stability of the tilt motion, it is more stable that the tilt guide 11 which provides rotation route for tilting is mounted to the linear translation block 32 which realizes the tilt motion rather than to the tilt block 12 where the tilt motion is realized.

Hereinafter, explanations will be made according to dispositions and connections shown in the drawings, but even in the second rotation positioner 2 and the linear positioner 3 dispositions may also be changed.

Meanwhile, referring to FIG. 4, the linear translation block 32 may include a tilt power transmitting unit 321 for transmitting power for rotating the tilt block 12 along the tilt guide 11to the tilt block 12, and the tilt block 12 of the first rotation positioner 1 may include a tilt portion 122 to which the tilting power is transmitted. In more detail, the tilt power transmitting unit 321 may include a rotation gear 3211, and the tilt portion 122 may include a tilt gear which is engaged with the rotation gear 3211 to receive the power so that the tilt block 12 rotates about the first axis a1.

As such, by the tilt power transmitting unit 321 provided to the linear translation block 32 and the tilt portion 122 provided to the first rotation positioner 1, the tilt motion of the first rotation positioner 1 relative to the linear translation block 32 can be realized. This will be explained again with the explanation of the linear translation block 32.

At this time, in case that connections are made in the sequence of the gantry 200, the second rotation positioner 2, and the first rotation positioner 1 like the gantry positioning device 100 according to an embodiment of the present invention, the first axis a1 related to the tilt motion may be defined with respect to the fixed supporter 300 since it is not affected by the wag motion of the second rotation positioner 2.

To the contrary, in case that connections are made in the sequence of the gantry 200, the first rotation positioner 1, and the second rotation positioner 2, the first axis a1 related to the tilt motion of the first rotation positioner 1 is affected by the wag motion of the second rotation positioner 2, so it may be defined with respect to the gantry 200 which is affected by the wag motion. This will be explained later with the explained of another embodiment of the present invention.

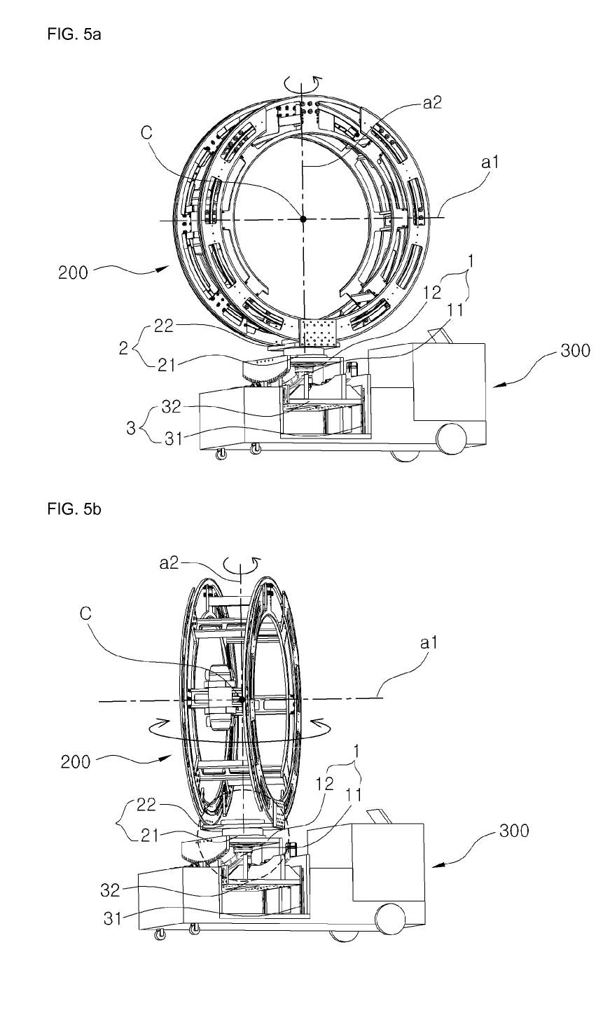

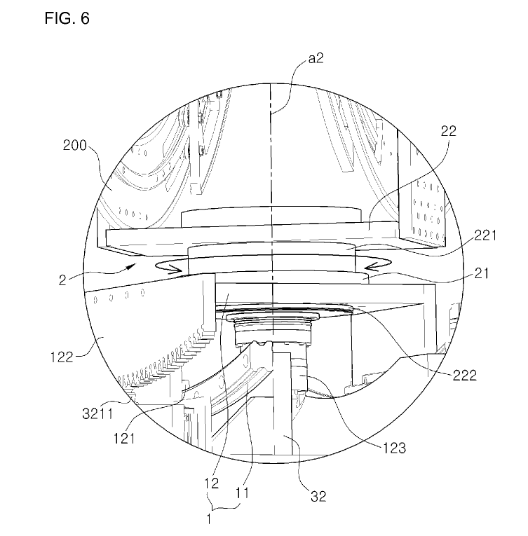

Hereinafter, the second rotation positioner 2 will be explained. FIG. 5a and FIG. 5b are drawings showing an example of a wag motion for waging a gantry of a gantry positioning device of FIG. 2, and FIG. 6 is an enlarged view of a portion depicted by a dotted line in FIG. 5b.

Referring to FIG. 5a and FIG. 5b, the second rotation positioner 2 rotates the gantry 200 about a second axis a2. At this time, the second axis a2 may be an axis passing the iso-center of the gantry 200 in a vertical direction. Here, the vertical direction may be a vertical direction a2 with respect to the gantry 200. That is, the vertical direction a2 with respect to the gantry 200 is not fixed when it is seen from the supporter 300 but moves and changes together with the rotation of the gantry 200.

That is, as shown in FIG. 5a and FIG. 5b, the wag motion of the gantry 200 may be realized through the second rotation positioner 2. In addition, the wag motion is realized by the rotation of the gantry 200 about the second axis a2, and since the second axis a2 passes the iso-center C of the gantry 200, the iso-center C of the gantry 200 may be fixed during the wag motion. That is, through the second rotation positioner 2, different from the conventional art, the gantry 200 may undergo the wag motion while the gantry 200 maintains the iso-centric state without movement of the iso-center C only through one-axis movement using only one freedom of degree.

In addition, referring to FIG. 5a, FIG. 5b, and FIG. 6, the second rotation positioner 2 may connect the gantry 200 and the tilt block 12 with each other such that the gantry 200 is rotatable about the second axis a2. For example, the second rotation positioner 2 may be directly connected to the gantry 200 at a top portion thereof so as to allow the gantry 200 to undergo the wag motion, and may be connected to the tilt block 12 of the first rotation positioner 1 at a bottom portion thereof so as to receive the tilt motion from the tilt block 12 thereby being tilted together with the gantry 200.

Meanwhile, referring to FIG. 5a, FIG. 5b, and FIG. 6, the second rotation positioner 2 may include a wag guide 21 which is formed to the first rotation positioner 1 so as to guide the rotation about the second axis a2, and may include a wag block 22 which is connected to the wag guide 21 so as to be rotatable about the second axis a2 and is connected to the gantry 200. As an example, referring to FIG. 6, the wag guide 21 may be formed to the tilt block 12 of the first rotation positioner 1. Further, the wag block 22 may include a wag coupling portion 221 which is engaged with the wag guide 21 so as to be movable along the wag guide 21.

That is, the wag block 22 which is connected to the gantry 200 is rotatably connected to the wag guide 21 via the wag coupling portion 221, the wag motion about the second axis a2 can be realized to the gantry 200 through the wag block 22. In addition, since the second axis a2 is an axis passing the iso-center C of the gantry 200, the wag motion of the gantry 200 can be realized by one axis operation using one freedom of degree in a state that the iso-center C of the gantry 200 is being fixed to one point.

In addition, as an example, the tilt block 12 of the first rotation positioner 1 may include a wag power transmission unit 123 transmitting power for rotating the wag block 22 about the second axis a2 along the wag guide 21 to the wag block 22, and the wag block 22 of the second rotation positioner 2 may include a wag portion 222 to which the power is transmitted.

Referring to FIG. 6, the wag portion 222 and the wag coupling portion 221 may be parts of the same member. That is, the wag portion 22 and the wag coupling portion 221 may be a single member which has not only a portion being rotatably engaged with the wag guide 21 but also a portion being receiving power from the wag power transmission unit 123.

At this time, in case that connections are made in the sequence of the gantry 200, the second rotation positioner 2, and the first rotation positioner 1 like the gantry positioning device 100 according to an embodiment of the present invention, the second axis a2 related to the wag motion of the second rotation positioner 2 is affected by the tilt motion of the first rotation positioner 1, so it may be defined with respect to the gantry 200 which is affected by the tile motion together. That is, the second axis a2 is not fixed when it is seen from the supporter 300 but is varied to be parallel with the vertical direction of the gantry 200 depending on the tilt motion. The reason of this is opposite to the reason why the lateral direction defining the first axis al is defined with respect to the supporter 300.

Meanwhile, in case that connections are made in the sequence of the gantry 200, the first rotation positioner 1, and the second rotation positioner 2, the second axis a2 related to the wag motion of the second rotation positioner 2 is not affected by the tilt motion of the first rotation positioner 1, so it may be defined with respect to the fixed supporter 300. This will be explained later as a gantry positioning device according to another embodiment of the present invention.

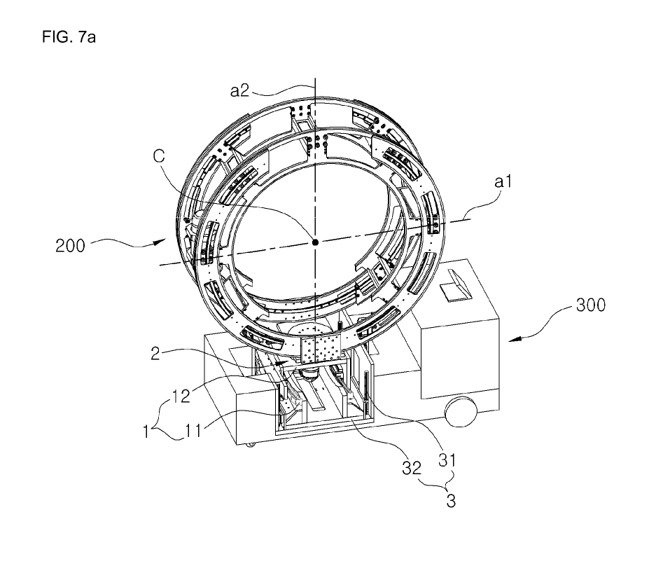

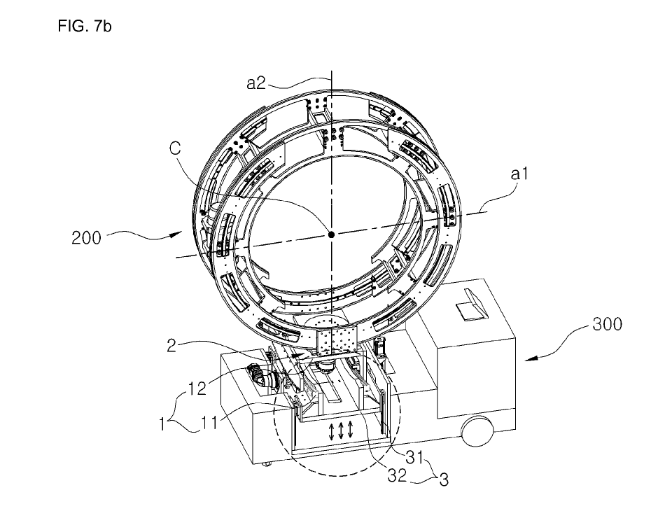

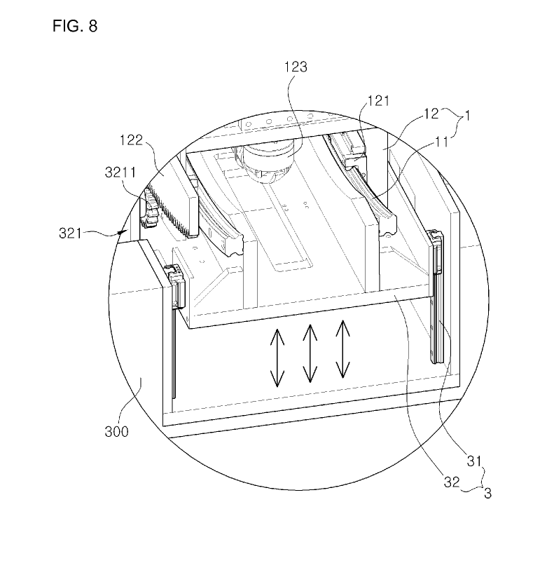

Hereinafter, the linear positioner 3 will be explained. FIG. 7a and FIG. 7b are drawings showing an example of a linear translation for translating vertically a gantry of a gantry positioning device of FIG. 2, and FIG. 8 is an enlarged view of a portion depicted by a dotted line in FIG. 7b.

Referring to FIG. 7a and FIG. 7b, the linear positioner 3 translates the gantry 200 in a vertical direction with respect to the supporter 300. That is, as shown in FIG. 7a and FIG. 7b, the linear translation motion of the gantry 200 can be realized by the linear positioner 3.

In addition, referring to FIG. 7a, FIG. 7b, and FIG. 8, the linear positioner 3 may connect the first rotation positioner 1 and the supporter 300 with one another such that the gantry 200 is vertically movable.

As an example, the linear positioner 3 may include a linear translation guide 31 which is formed to the supporter 300 so as to guide the movement along a vertical direction, and may include the linear translation block 32 which is connected to the linear translation guide 31 so as to be movable in a vertical direction and to which the tilt guide 11 of the first rotation positioner 1 is coupled.

Referring again to FIG. 3a, FIG. 3b, and FIG. 4, by mounting the tilt guide 11 to the linear translation block 32, the first rotation positioner 1 is rotatably connected to the linear positioner 3 through the connections with the tilt block 12 and the tilt guide 11 so as to realize the tilt motion.

Further, referring to FIG. 4 and FIG. 8, the linear translation block 32 includes the tilt power transmitting unit 321 which transmits power for rotating the tilt block 12 along the tilt guide 11 to the tilt block 12, and the tilt block 12 of the first rotation positioner 1 may include the tilt portion 122 receiving this power. In more detail, the tilt power transmitting unit 321 may include a rotation gear 3211 for transmitting power. In addition, the tilt power transmitting unit 321 may include a rotation motor for driving the rotation gear 3211. The rotation gear 3211 may be a spur gear or a pinion gear. The tilt portion 122 may include a tilt gear which is engaged with the rotation gear 3211 so as to receive the power so that the tilt block 12 can rotate about the first axis a1.

Exemplarily, referring to FIG. 4 and FIG. 8, the tilt gear is formed at a lower portion of the tilt portion 122 along an arc having a center on the first axis a1 and is engaged with the rotation gear 3211, and by this engagement, the tilt portion 122 can receive power generated by the rotation motor of the tilt power transmitting unit 321, and the tilt motion of the tilt block 12 can be realized. That is, by the tilt power transmitting unit 321 provided to the linear translation block 32 and the tilt portion 122 provided to the first rotation positioner 1, the tilt motion with respect to the linear translation block 32 can be realized to the first rotation positioner 1.

In addition, referring to FIG. 2 to FIG. 8, the gantry positioning device 100 according to an embodiment of the present invention can be disposed above the supporter 300, and the gantry 200 may be disposed above the gantry positioning device 100.

That is, the supporter 300 plays a role of a base for stably supporting the gantry 200 which is connected to an upper portion of the gantry positioning device 100.

In addition, the gantry positioning device 100 supports the gantry 200 from a bottom side and positions the same, stability in an operation can be enhanced, when compared to a conventional type in which the gantry is supported in a cantilevered type, so initial cost for securing the stability or maintenance cost can be reduced, and in terms of space, total space of the device can be substantially reduced compared to the conventional cantilevered type which has a lateral connection so that spatial utilization can be improved.

In addition, the gantry positioning device 100 may include a controller for controlling the above-stated operations. The controller may be provided by being mounted to the gantry positioning device 100, or may be disposed to be separated from the gantry positioning device 100 to send a control signal and a control signal receiver is provided to the gantry positioning device 100 so as to be remote-controlled.

Operations of the gantry positioning device 100 will be explained hereinafter. Referring to FIG. 3a, FIG. 3b, and FIG. 4, the tilt motion of the gantry 200 by the gantry positioning device 100 is explained. For example, if the rotation motor of the tilt power transmitting unit 321 is operated by the controller for controlling the gantry positioning device 100, the power is transmitted to the rotation gear 3211 and the power is also transmitted to the tilt block 12 through the tilt portion 122 which is engaged with the rotation gear 3211. By this power, the tilt block 12 is rotated along the tilt guide 11 about the first axis a1. By the rotation of the tilt block 12 about the first axis a1, the tilt motion occurs to the second rotation positioner 2 connected to the tilt block 12 and the gantry connected to the second rotation positioner 2.

Since the tilt motion is performed about the first axis a1 passing the iso-center C of the gantry 200 [symbol] the iso-center C, the iso-center C of the gantry 200 does not move and is maintained to be fixed as an iso-centric state.

Subsequently, referring to FIG. 5a, FIG. 5b, and FIG. 6, the wag motion of the gantry 200 realized by the gantry positioning device 100 is explained. For example, if the rotation motor of the wag power transmission unit 123 is operated by the controller for controlling the gantry positioning device 100, the power is transmitted to the wag portion 222 and thereby the wag coupling portion 221 rotates along the wag guide 21 about the second axis a2. By the rotation of the wag block 22 about the second axis a2, the wag motion is realized to the gantry 200 which is connected to the wag block 22.

Since the wag motion is performed about the second axis a2 passing the iso-center C of the gantry 200, the iso-center C of the gantry 200 does not move and is maintained to be fixed as an iso-centric state.

Subsequently, referring to FIG. 7a, FIG. 7b, and FIG. 8, the linear translation motion of the gantry 200 realized by the gantry positioning device 100 is explained. As an example, if a linear translation power transmission unit (not shown) for transmitting power for vertical movement is operated by the controller for controlling the gantry positioning device 100, the power is transmitted to the linear translation block 32 and the linear translation block 32 can be moved in a vertical direction. The linear translation power transmission unit may be directly connected to the linear translation block 32 to transmit the power, but it is not limited thereto, and for example, may be connected to the first rotation positioner 1 which is connected to the linear translation block 32, the second rotation positioner 2, or the gantry 200 so as to transmit power for vertical movement to the linear translation block 32. By the vertical linear translation of the linear translation block 32, the linear translation motion can be realized to the first rotation positioner 1 which is connected to the linear translation block 32, the second rotation positioner 2 which is connected to the first rotation positioner 1, and the gantry 200 which is connected to the second rotation positioner 2.

Hereinafter, a gantry positioning device according to another embodiment of the present invention will be described. Explanations will focus on the differences from the above-described the gantry positioning device 100, and the same reference numeral will be used for the same parts with the gantry positioning device 100 and repeated explanations will be omitted or simplified.

Not shown in the drawings, in the gantry positioning device according to another embodiment of the present invention, the tilt block 12 of the first rotation positioner 1 may be directly connected to the gantry 200. In addition, the second rotation positioner 2 may be connected to the first rotation positioner 1 so that the gantry 200 and the first rotation positioner 1 can be rotatable about the second axis a2. Accordingly, the lateral defining the axial direction of the first axis a1 may be the lateral direction with respect to the gantry 200 and the vertical direction defining the axial direction of the second axis a2 may be the vertical direction with respect to the supporter 300.

That is, the gantry positioning device according to another embodiment of the present invention differs from the gantry positioning device 100 in terms of the connections of parts. That is, the gantry positioning device 100 has the connections in the sequence of the gantry 200, the second rotation positioner 2, and the first rotation positioner 1, but the gantry positioning device according to another embodiment of the present invention may have the connections in the sequence of the gantry 200, the first rotation positioner 1, and the second rotation positioner 2. In other words, different from the gantry positioning device 100 in which the second rotation positioner 2 is directly connected to the gantry 200, the first rotation positioner 1 is directly connected to the gantry 200 in the gantry positioning device according to another embodiment of the present invention.

In case of the gantry positioning device according to another embodiment of the present invention, the second axis a2 related to the wag motion of the second rotation positioner 2 is not affected by the tile motion of the first rotation positioner 1, so it may be defined with respect to the supporter 300 which is fixed. In addition, the first axis a1 related to the tilt motion of the first rotation positioner 1 is affected by the wag motion of the second rotation positioner 2, so it may be defined with respect to the gantry 200 which is affected by the wag motion together.

As such, in case the first rotation positioner 1 is connected to the gantry 200 and the second rotation positioner 2 is connected to the first rotation positioner 1, since the wag motion of the second rotation positioner 2 is realized not only to the gantry 200 but also to the first rotation positioner 1, the first axis a1 is not fixed when it is seen from the supporter 300 but is changed with the wag motion along a direction parallel with the lateral direction of the gantry 200. Therefore, the lateral direction defining the axis direction of the first axis a1 may be the lateral direction with respect to the gantry 200 instead of the supporter 300. In addition, in this connection, since the tile motion of the first rotation positioner 1 is realized only to the gantry 200 but not to the second rotation positioner 2, the vertical direction defining the axial direction of the second axis a2 may be the vertical axis with respect to the supporter 300 which is not affected by the tilt motion instead of the gantry 200.

For reference, the linear positioner 3 may be formed to connect the second rotation positioner 2 and the supporter 300 together, but it is not limited thereto, and it may be formed to connect the first rotation positioner 1 and the second rotation positioner 2 together.

Hereinafter, an imaging device 1000 according to an embodiment of the present invention will be explained. For ease of explanation, the same reference numeral will be used for the same parts with the above-mentioned embodiments and repeated explanations will be omitted or simplified.

Referring to FIG. 2, the imaging device 1000 may include a gantry positioning device, the gantry 200, and the supporter 300. At this time, the gantry positioning device may be the gantry positioning device 100 or the gantry positioning device according to another embodiment of the present invention.

In addition, as shown in FIG. 2, the gantry 200 may have a ring shape. Exemplarily, the ring shape of the gantry 200 may be an O-shape, a C-shape, an annular ring shape, a circular plate shape having a circular through hole at a center thereof, or the like.

In addition, not shown in the drawings, the gantry 200 may include a gantry operation unit 150 which is rotatable along a circumferential direction of the ring shape. The gantry operation unit may include a light source 151 for emitting light and a light detector 152 for receiving light emitted from the light source. The light source 151 and the light detector 152 may be disposed to face each other.

Exemplarily, the light source 151 and the light detector 152 are disposed to face each other at opposite sides of the perforated center portion of the ring-shaped gantry 200, so an object which will be scanned is set at the perforated center portion of the gantry 200 and then light emission is performed. At this time, the gantry operation unit 150 rotates along a circumferential direction, so three-dimensional images as well as two-dimensional images can be obtained.

In particular, in case that the light source 151 and the light detector 152 are disposed such that the iso-center C of the gantry 200 is disposed therebetween, if the object is set at the iso-center C of the gantry 200, the position of the iso-center C of the gantry 200 is not changed even though the tilt or wag motion occurs. Accordingly, light emission for the object can be fixed to the iso-center C so as to realize the iso-centric state while the tilt or wag motion occurs, so precise image scanning can be stably performed in various directions.

Light emitted from the light source 151 may be light having a X-ray wavelength. That is, the light source 151 may emit X-ray beam, and the light detector 152 may receive the X-ray beam. By using the X-ray beam, the imaging device 1000 may be adopted to the two-dimensional or three-dimensional computed tomography.

In addition, the supporter 300 may be mobile. The imaging device 1000 may realize the vertical linear translation, the tilt motion, and the wag motion through the control of the gantry positioning device, and horizontal translation can be performed by the movement of the supporter 300.

Further, the supporter 300, as shown in FIG. 2, may include an indentation for receiving the gantry positioning device. This indentation may be formed by being indented downwardly, and for example the linear positioner 3 of the gantry positioning device may be disposed at the indentation. For example, the linear positioner 3 may be disposed in such a way that the linear translation guide 31 of the linear positioner 3 may be disposed along the side wall of the indentation and the linear translation block 32 may be connected thereto.

Since the indentation is provided to the supporter 300, the gantry positioning device can be more efficiently and stably disposed, and an overall volume of the imaging device 1000 can be reduced to enhance a spatial applicability.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. A gantry positioning device for connecting a gantry and a supporter together of an imaging system, comprising:
a linear translation positioner for translating the gantry vertically with respect to the supporter;
a first rotation positioner for rotating the gantry about a first axis which passes horizontally an iso-center of the gantry with respect to the supporter; and
a second rotation positioner for rotating the gantry about a second axis which passes vertically the iso-center of the gantry with respect to the supporter,
wherein the first rotation positioner comprises:
a tilt guide which is formed along an arc having a center on the first axis; and
a tilt block which is connected to the tilt guide so as to be rotatable about the first axis.

2. The gantry positioning device of claim 1, wherein the second rotation positioner connects the gantry and the tilt block such that the gantry is rotatable about the second axis, the horizontal direction is a horizontal direction with respect to the supporter, and the vertical direction is a vertical direction with respect to the gantry.

3. The gantry positioning device of claim 2, wherein the second rotation positioner comprises:
a wag guide which is formed to the first rotation positioner for guiding a rotation about the second axis; and
a wag block which is connected to the wag guide so as to be rotatable about the second axis and is connected to the gantry.

4. The gantry positioning device of claim 2, wherein the linear translation positioner connects the first rotation positioner and the supporter together such that the gantry is movable along the vertical direction.

5. The gantry positioning device of claim 4, wherein the linear translation positioner comprises:
a linear translation guide which is provided to the supporter to guide a movement along the vertical direction; and
a linear translation block which is connected to the linear translation guide so as to be movable along the vertical direction and to which the tilt guide is provided.

6. The gantry positioning device of claim 5, wherein the linear translation block comprises a tilt power transmission unit for transmitting power for rotating the tilt block along the tilt guide to the tilt block, and the tilt block comprises a tilt portion which receives the power.

7. The gantry positioning device of claim 6, wherein the tilt power transmission unit comprises a rotation gear for transmitting the power, and the tilt portion comprises a tilt gear which is engaged with the rotation gear to receive the power so that the tilt block rotates about the first axis.

8. An imaging device comprising:
a gantry;
a supporter; and
a gantry positioning device for connecting the gantry and the supporter together of an imaging system
wherein the gantry positioning device comprising:
a linear translation positioner for translating the gantry vertically with respect to the supporter;
a first rotation positioner for rotating the gantry about a first axis which passes horizontally an iso-center of the gantry with respect to the supporter; and
a second rotation positioner for rotating the gantry about a second axis which passes vertically the iso-center of the gantry with respect to the supporter,
wherein the first rotation positioner comprises:
a tilt guide which is formed along an arc having a center on the first axis; and
a tilt block which is connected to the tilt guide so as to be rotatable about the first axis.

9. The imaging device of claim 8, wherein the gantry comprises a gantry operation unit, and the gantry operation unit comprises a light source emitting light and a light detector receiving the light, and the light source and the detector are disposed to face each other.

10. The imaging device of claim 8, wherein the supporter is movable.
